(19) United States
(12) Patent Application Publication
(10) Pub. No.: US 2002/0001565 A1
(43) Pub. Date: Jan. 3, 2002

(54) Radioprotective agents

(76) Inventor: Alla Shapiro, Rockville, MD (US)

Correspondence Address: Karen L. Orzechowski, Liniak, Berenato, Longacre & White, LLC, Suite 240, 6550 Rock Spring Drive, Bethesda, MD, 20817, US

(21) Appl. No.: 09/916,257

(22) Filed: Jul. 30, 2001

Related U.S. Application Data
(63) Continuation of application No. PCT/US01/18772, filed on Jun. 12, 2001.
(60) Provisional application No. 60/211,375, filed on Jun. 14, 2000.

(51) Int. Cl. A61K 51/00; A61K 31/7048; A61K 31/353

(52) U.S. Cl. 424/1.11; 514/27; 514/456

(57) ABSTRACT

A non-toxic and effective isoflavone radioprotective agent for treating or preventing effects and damage due to radiation exposure, or increasing the survivability to a lethal dose of radiation. The isoflavone can be administered orally, subcutaneously, intramuscularly, intravenously, transdermally, intranasally, or rectally. The isoflavone is administered chronically, and/or before, during and/or after radiation exposure. These radioprotective agents can be used to protect personnel exposed to radioactive substances.

### CROSS REFERENCE TO RELATED APPLICATIONS

This application claims benefit under 35 USC §119(e) of U.S. Provisional Application Serial No. 60/211,375, filed Jun. 14, 2000.

### FIELD OF THE INVENTION

The present invention relates to a method for protecting mammals, in particular humans, from toxic effects of radiation. The present invention further relates to the use of radioprotective agents to prevent and/or treat serious or lethal damage to living cells, tissues and/or living organisms due to radiation exposure. The invention further relates to the use of isoflavones, in particular genistein, to prevent or treat damage from acute or chronic exposure to radiation. The invention also relates to the protection of normal tissues during diagnostic and therapeutic radiation exposure.

### BACKGROUND OF THE INVENTION

The increased use of radionuclides in diagnostic and therapeutic nuclear medicine as well as the presence of man-made and naturally occurring radioactivity in the environment has created the need for radioprotective agents for protection of living cells, tissues and living organisms before, during, and after exposure to radiation.

Radioprotective agents, also known as radioprotectors, are defined as agents that protect cells or living organisms from deleterious cellular effects of exposure to ionizing radiation. These deleterious cellular effects include damage to cellular DNA, such as DNA strand break, disruption in cellular function, cell death and/or carcinogenisis. The mechanism of this protective effect may at least partially be due to radical scavenging properties and cell cycle modulating properties of the radioprotective agents.

The potential utility of these agents in protecting against exposure to environmental radiation, as well as in cancer radiation therapy, has long been recognized. These agents, administered prior to, during, and/or after exposure to radiation, would eliminate or reduce the severity of deleterious cellular effects caused by exposure to environmental ionizing radiation such as resulting from a nuclear explosion, a spill of radioactive material, close proximity to radioactive material and the like.

In addition, these agents are believed to provide a selective protection of normal cells, and not of cancer cells, during cancer radiation therapy. For example, these agents, administered to the cancer patient prior to or during radiation therapy, will be absorbed by normal, non-cancer cells to provide a protective effect. However, the radioprotective agents will be absorbed to a lesser extent, if at all, by tumor cells due to the poor vascularity and other known biological differences between normal and tumor cells. Therefore, the radioprotective agents would provide a selective protective effect on the normal cells as compared to tumor cells and would eliminate or reduce the severity of deleterious or other detrimental cellular effects of radiation therapy on normal cells. Furthermore, some radioprotective agents may act as prodrugs and require activation by cellular enzymatic processes which are not fully operative in the cancer cell. These agents, even if absorbed in a similar concentration in normal and cancer cells, will only be activated in cells with normal enzymatic processes and not in cancer cells. These prodrug radioprotective agents would be activated to provide a selective protective effect only in normal cells and would thus eliminate or reduce the severity of deleterious or detrimental cellular effects of radiation therapy on normal cells.

Radioprotective agents thus are useful in eliminating or reducing the severity of deleterious cellular effects in normal cells caused by environmental exposure to radiation, cancer radiation therapy and diagnostic tests utilizing radiation.

For example, the treatment of malignant tumors through the use of radiation is often limited due to damage to non-tumor cells. Damage to the non-tumor cells can exceed the effectiveness of the radiation therapy. The dominant consideration in establishing radiation doses for cancer radiotherapy is the assessment of tolerance of the most radiosensitive normal tissue or organ in the treatment field. This assessment, together with the expected radiation dose required to eradicate a tumor determines the feasibility of the treatment strategy, and whether a cure or palliation is to be attempted. Often, the maximum tolerable doses are insufficient to eradicate the tumor. Thus, the use of a radioprotective agent would greatly increase the tolerable dose, and therefore the prospects for eradication of tumors and treatment of the cancer.

Attempts have been made to create radioprotective agents for administration to living subjects. However, problems have arisen with the potency of the radioprotective agents; delivery to the cells, tissue or organs to be protected; and the toxicity of the radio protective agent to not only the cells, tissue or organs, but also to the living subject. Therefore there remains an acute need for non-toxic and effective radioprotectors with acceptable and/or convenient routes of administration.

Additionally, therapy and diagnostic tests utilizing radiation are withheld from pregnant women, women who may be pregnant, and women capable of becoming pregnant to avoid harming the fetus in utero. This can often preclude necessary treatment or diagnosis for these women. Accordingly, radioprotective agents that are non-toxic and highly effective can be administered to such women so as to confer protection on the women and any possible fetus above and beyond any conventional mechanical radiation shielding device. This can also provide a level of safety to those women nursing their infants.

It has now been discovered that an isoflavone compound, in particular genistein, can be taken orally and is capable of providing radioprotection from lethal effects of radiation exposure, either prophylactically and/or after exposure and significantly diminishing damage caused by sublethal doses of radiation, such as used in medical procedures and diagnostic tests.

Accordingly, it is a primary object of this invention to provide a method for reducing or preventing damage to the living organisms caused by radiation by the administration of isoflavone compounds before, during or after exposure to radiation. It is also an object of this invention to provide a method for increasing survivability from lethal doses of radiation exposure by the administration of isoflavone compounds.

### SUMMARY OF THE INVENTION

The present invention is directed to a method for increasing the survivability of humans or animals from a lethal dose of irradiation, the method comprising of oral administration to a human or other species before or immediately after radiation exposure an effective amount of an isoflavone compound, particularly genistein. In particular, the present invention is directed to a nontoxic and highly effective radioprotective agent that can be administered orally.

The present invention is further directed to protection of normal cells and tissues in a mammal from therapeutic or diagnostic radiation exposure by administration of an isoflavone compound. This enables larger, more effective, doses of radiation to be given to the patient.

The invention further relates to an isoflavone derived from soy that imparts radiation resistance. Additionally, the radioprotective agent of the present invention can be administered chronically.

### DETAILED DESCRIPTION

The present invention provides a method of protecting living cells, tissues and organisms from serious or fatal damage or deleterious cellular effects caused by acute or chronic exposure to radiation and for the protection of normal cells, tissues and organisms during radiation treatment in patients.

Ionizing radiation is high-energy radiation, such as an X-ray or a gamma ray, which interacts to produce ion pairs in matter. Exposure to ionizing radiation may occur as the result of environmental radiation, such as resulting from a nuclear explosion, a spill of radioactive material, close proximity to radioactive material and the like. More commonly, exposure to ionizing radiation may occur as the result of radiological medical procedures such as radiation therapy for various types of cancers or for diagnostic purposes such as in diagnostic x-rays, computer aided tomography (CAT) scans, mammograms, radionuclide scans and the like.

The radioprotective agents of the present invention can be used to minimize or prevent the damage from solar radiation exposure experienced by astronauts, pilots, other flight personnel and frequent fliers. The radioprotective agents can also be utilized in protecting from accidental radiation exposure from nuclear power facilities, other radiation generating facilities including those for food irradiation, or as a result of detonation of an atomic bomb of other device that releases radiation. Also, they can be used to confer protection to those personnel involved with clean up of such radiation accidents or disposal facilities. The radioprotective agents of the present invention are also of use in reducing the toxic effects of inhaled or ingested radionuclides and in reducing toxicity from radiation produced by electronic devices of non-ionizing nature of radiation: such as cellular telephones, and microwaves. Rapidly growing interventional radiologic procedures such as dilatation of stenosed vessels, recanalization or vascular angioanastomoses would also benefit from the use of radioprotectors.

Deleterious cellular effects caused by exposure to radiation include damage to cellular DNA, such as DNA strand break, disruption in cellular function, the ability to repair damage caused by free radicals, cell death, tumor induction, radiation induced thyroid cancer and leukemia and the like. These deleterious cellular effects can lead to secondary tumors, bone marrow suppression, kidney damage, peripheral nerve damage, gastrointestinal damage and the like. For example, in cancer radiation therapy, the exposure to radiation is intended to cause cell death in the cancer cells. Unfortunately, a large part of the adverse events associated with the therapy is caused by these deleterious cellular effects of the radiation on normal cells as opposed to cancer cells.

The present invention provides a method which protect cells and living organisms from deleterious cellular effects by preventing or eliminating these effects or by reducing their severity. According to the present invention, living organisms to be protected can be exposed with an isoflavone compound prior to or during exposure of the cell to radiation. The cells may be directly treated by isoflavone compounds, such as by applying a solution of an isoflavone compound of the invention to the cell or by administering an isoflavone compound of the invention to a mammal. The compounds of the present invention thus can provide a protective effect in the cell and living organisms which eliminates or reduces the severity of the detrimental cellular effects which would otherwise be caused by the exposure.

The radioprotective agents of the present invention enables survival of living organisms in otherwise lethal conditions.

More particularly, the present invention provides a method of protecting non-cancer, or normal, cells of a mammal from deleterious cellular effects caused by exposure of the mammal to ionizing radiation. As used herein, the term “mammal” refers to warm-blooded animals such as mice, rats, dogs and humans. The compounds of the present invention provide a protection of normal cells during exposure to radiation, such as during radiation therapy or diagnostic procedures such as x-rays and CAT scans. The cancer cells, if protected at all, are protected to a lesser extent than normal cells. The present invention provides a method whereby the deleterious cellular effects on non-cancer cells caused by exposure of the mammal to radiation are eliminated or reduced in severity or in extent

Isoflavone compounds particularly useful in the present invention include compounds having the general formula:

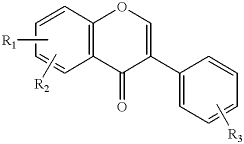

wherein R₁, R₂ and R₃ are independently selected from the group consisting of hydrogen, hydroxyl and alkoxy.

Isoflavone compounds of interest include genistein, genistin, daidzein, daidzin, glycitein, glycitin, biochannin A, formononetin, O-desmethyangolensin, equol and the like, their glucosides and derivatives, and/or mixtures thereof Of particular importance is genistein, also known as 5,7-dihydroxy-3-(4-hydroxyphenyl)-4H-1 benzopyran-4-one or 4′,5,7-trihydroxyisoflavone.

These isoflavone compounds exhibit antioxidant properties and estrogenic activity and can act as a tyrosine kinase inhibitor and/or an angiogenesis inhibitor. These isoflavone compounds can also act to lower LDL cholesterol concentration and as a vasodilitory agent.

The isoflavone compounds can be derived from any suitable source such as soy, legumes, clover and the like using any of the techniques well known to one of ordinary skill in the art.

According to the present invention the isoflavone compound of the invention is administered to the mammal prior to, during, or immediately after exposure to the radiation. In one embodiment of the invention, the isoflavone compound can be administered on a continuing basis for protection against anticipated exposure to doses of acute radiation and for continuing protection against exposure to doses of chronic radiation. In another embodiment of the invention, the isoflavone compound of the present invention is administered within two weeks before, during, and/or within two weeks after exposure to radiation. In another embodiment, the isoflavone compound of the present invention is administered both within 4 days prior to exposure and within 4 days after exposure.

The compounds of the invention should be administered to the human or other animal prior to irradiation in an amount which is effective for diminishing damage to the respiratory, gastrointestinal and the hematopoietic systems after sublethal irradiation or for increasing the survival rate after lethal irradiation. The compounds are also effective when administered immediately after exposure to radiation, i.e. up to 30-60 minutes. Prophylactic oral, parenteral or topical administration of genistein would protect the military personnel or other living organisms exposed to radiation. This activity makes the invention of special utility to workers in the nuclear industry and to the military where personnel may be exposed to radiation.

The other suggested dosing regimens would include multiple doses of oral genistein given four days prior and four days following the exposure to radiation.

Calculation of the dosage to be administered to the subject can be readily calculated by one of ordinary skill in the art. For example, the approximate human doses extrapolated from the preclinical data obtained in mice is approximately 29 mg/kg/bw.

According to the present invention, administration to a patient of an isoflavone compound prior to or during radiation therapy will provide a selective protection of non-cancer cells of the patient in preference to cancer cells. The deleterious cellular effects on non-cancer cells caused by treatment of the patient with ionizing radiation are thus eliminated or reduced in severity or in extent.

A protective amount of an isoflavone compound refers to that amount which is non-toxic and effective, upon single or multiple dose administration to a mammal or patient, in eliminating or reducing in severity or in extent the deleterious cellular effects caused by exposure to or treatment with ionizing radiation. A protective amount of an isoflavone compound also refers to that amount which is effective, upon single or multiple dose administration to humans and other living organisms, in eliminating or reducing in severity or in extent the destructive cellular effects caused by exposure to ionizing radiation.

A protective amount for administration to a mammal or a patient can be readily determined by one of ordinary skill in the art, by the use of known techniques and by observing results obtained under analogous circumstances. In determining the protective amount or dose, a number of factors are considered by one skilled in the art, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances.

The isoflavone compounds of the present invention may be administered as single doses or as multiple doses and are ordinarily administered prior to and/or during exposure to radiation. Generally, where a compound of the present invention is administered in conjunction with radiation therapy, the compound of the present invention will be administered in single or multiple doses prior to radiation therapy following a schedule calculated to provide the maximum selective protective effect during radiation therapy. Generally, where a compound of the present invention is administered in conjunction with other therapeutic agents, the compound of the present invention will be administered in single or multiple doses prior to and during therapy following a schedule calculated to provide the maximum selective protective effect during therapy.

The details of the dosing schedule for the compounds of the present invention necessary to provide the maximum selective protective effect upon exposure to ionizing radiation can be readily determined by one skilled in the art by the use of known techniques and by observing results obtained under analogous circumstances.

A protective amount of an isoflavone compound for administration to a mammal or patient will vary depending upon the amount of radiation exposure and the time period of radiation exposure, with the upper limit of the isoflavone compound limited by the toxicity of a large dose. A larger dose of an isoflavone compound will be required for lethal radiation exposure, while a lower dose can be used where the radiation exposure is sub-lethal or chronic. For example, the isoflavone compounds of the present invention can be administered from about 100 mg/kg of body weight per day to about 400 mg/kg per day. Preferred amounts are expected to vary from about 29 mg/kg to about 400 mg/kg for mammals.

An isoflavone compound can be administered to a mammal, a healthy individual, or a patient in any form or mode which makes the compound bioavailable in effective amounts, including oral and parenteral routes. For example, the isoflavone compounds of the present invention can be administered orally, subcutaneously, intramuscularly, intravenously, transdermally, intranasally, rectally, and the like. Oral administration is generally preferred. One skilled in the art of preparing formulations can readily select the proper form and mode of administration depending upon the particular characteristics of the compound selected the disease state to be treated, the stage of the disease, and other relevant circumstances.

The compounds can be administered alone or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. The compounds of the invention, while effective themselves, may be formulated and administered in the form of their pharmaceutically acceptable acid addition salts for purposes of stability, convenience of crystallization, increased solubility and the like.

In another embodiment, the present invention provides compositions comprising an isoflavone compound in admixture or otherwise in association with one or more inert carriers. Inert carriers can be any material which does not degrade or otherwise covalently react with an isoflavone compound of the present invention. Examples of suitable inert carriers are water; aqueous buffers, such as those which are generally useful in High Performance Liquid Chromatography (HPLC) analysis; organic solvents, such as acetonitrile, ethyl acetate, hexane and the like; and pharmaceutically acceptable carriers or excipients.

More particularly, the present invention provides pharmaceutical compositions comprising a therapeutically effective amount of an isoflavone compound of the present invention in admixture or otherwise in association with one or more pharmaceutically acceptable carriers or excipients.

The pharmaceutical compositions are prepared in a manner well known in the pharmaceutical art. The carrier or excipient may be a solid, semi-solid, or liquid material which can serve as a vehicle or medium for the active ingredient. Suitable carriers or excipients are well known in the art. The pharmaceutical composition may be adapted for oral or parenteral use and may be administered to the patient in the form of tablets, capsules, suppositories, solution, suspensions, or the like.

The compounds of the present invention may be administered orally, for example, with an inert diluent or with an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, wafers, chewing gums, transdermal delivery devices and the like.

The tablets, pills, capsules, troches and the like may also contain one or more of the following adjuvants: binders such as microcrystalline cellulose, gum tragacanth or gelatin; excipients such as starch or lactose, disintegrating agents such as alginic acid, Primogel™, corn starch and the like; lubricants such as magnesium stearate or Sterotex™; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

For the purpose of parenteral therapeutic administration, the compounds of the present invention may be incorporated into a solution or suspension. The amount of the inventive compound present in such compositions is such that a suitable dosage will be obtained.

The solutions or suspensions may also include the one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

In a further embodiment of the invention, the isoflavone of the present invention can be administered as a food supplement so as to confer continuing protection against radiation exposure such as that encountered by nuclear power workers, x-ray technicians, and the like. Use as a food supplement enables the radioprotective agent of the present invention to be given on a daily basis in the event of an unpredictable radiation event.

The utility of the compounds of the present invention may be demonstrated as radioprotective agents both in vitro and in vivo.

For example, the ability of cultured cells to form clones (colonies) may be evaluated as a function of exposure to X-ray dose. Cells are either not drug treated or are treated with a test agent 30 minutes prior to exposure. The degree of retention of ability to form clones after exposure, in comparison to untreated cells, is directly related to the protective effect of the drug. A typical experiment of this type may be carried out essentially as described by Snyder and Lachmann [Radiation Res. 120, 121 (1989)].

Alternatively, the production of DNA strand breaks upon exposure to X-ray dose may be evaluated. Cells are either not drug treated or are treated with a test agent about 30 minutes prior to exposure. The extent of DNA strand breakage after exposure, in comparison to that in untreated cells, is inversely related to the protective effect of the drug. A typical experiment of this type may be carried out essentially as described by Snyder [Int. J. Radiat. Biol. 55, 773 (1989)].

In addition, the survivability of mice exposed to whole body irradiation may be evaluated. Animals, either pre-treated with a test agent or untreated (Control Group), are exposed to whole body irradiation (1500 rads). Untreated control animals are expected to survive about 12-15 days. The degree of survivability of the treated animals, in comparison to the untreated controls, is directly related to the protective effect of the drug treatment. A typical experiment of this type may be carried out essentially as described by Carroll et al. [J. Med. Chem. 33, 2501 (1990)].

The production of DNA strand breaks in lymphocytes taken from treated animals exposed to whole body irradiation may be evaluated in comparison to untreated control. Alternatively, the viability and clonogenicity of bone marrow cells taken from treated animals exposed to whole body irradiation may be evaluated in comparison to untreated control as described by Pike and Robinson [J. Cell Physiol. 76, 77 (1970)].

In order to fully illustrate the nature of the invention, and the manner of practicing the same, the following examples are presented.

### EXAMPLE 1

The radioprotective potential of isoflavones, particularly genistein, was demonstrated in CD2F 1 male mice as measured by 30-day survival after exposure to a lethal dose of cobalt-60 radiation (9.5 Gy at 0.6 Gy/min). Control groups were administered saline and polyethylene glycol 400 (the vehicle for the genistein) prior to irradiation. Genistein (Sigma Chemical Co., St. Louis, Mo.) was administered 400 mg/kg either as a single dose or multiple dosages of 100 mg/kg each at various times ranging from 1 hour to 4 days before radiation, after irradiation, or both before and after radiation.

Forty-four percent of mice survived if they received genistein only before radiation, 0% if given genistein only after irradiation, and 69% survived if they received genistein before and after irradiation. This compares with 0% surviving in a control group given saline and 19% surviving if administered the genistein vehicle, polyethylene glycol 400. For mice receiving multiple oral administration of a lower dose of genistein (100 mg/kg), 0% survived if given genistein 4 days before irradiation, 0% after irradiation, and 50% survived when given 100 mg/kg genistein daily for 4 days before and 4 days after irradiation. A single dose of genistein (400 mg/kg) given orally 1 hr or 24 hr before 9.5 Gy radiation did not confer any measurable radioprotection. These experiments demonstrate that single or multiple oral doses of genistein protect mice from a lethal dose of ionizing radiation.

### EXAMPLE 2

In subsequent studies, using the procedures and mice of Example 1, a single subcutaneous dose of 100 or 400 mg/kg genistein was administered 24 hr before 9.5 Gy radiation. The mice exhibited 30-day survival rates of 69% and 81%, respectively. These experiments demonstrate that a subcutaneous dose of genistein protects mice from a lethal dose of ionizing radiation.

### EXAMPLE 3

The procedure of Examples 1 and 2 are followed using other isoflavones including genistein, genistin, daidzein, daidzin, glycitein, glycitin, biochannin A, formononetin, O-desmethylangolensin, and equol, their glucosides and derivatives, and mixtures thereof. Similar radioprotective effects are shown.

### EXAMPLE 4

Isoflavones of the present invention including genistein, genistin, daidzein, daidzin, glycitein, glycitin, biochannin A, formononetin, O-desmethylangolensin, and equol, their glucosides and derivatives, and mixtures thereof are given chronically to humans to treat or prevent effects from exposure to radiation. The isoflavone is administered orally, subcutaneously, intramuscularly, intravenously, transdermally, intranasally, and/or rectally. Similar radioprotective effects are shown.

### EXAMPLE 5

Genistein, genistin, daidzein, and/or mixtures thereof are administered to patients with malignant tumors in various locations that require radiation treatment in order to protect the healthy tissues and/or intensify the doses of radiation to the tumor for more effective eradication of tumor cells. These isoflavone compounds are administered before the planned radiation treatment, during and/or after the radiation treatment to selectively confer radioprotection to the healthy tissues of the patients.

### EXAMPLE 6

Military personnel prior to exposure to lethal and/or sublethal doses of radiation are administered prophylactically genistein, genistin, daidzein, or mixtures thereof either orally or by transdermal patch to confer radioprotection. Radioprotective effects are demonstrated.

### EXAMPLE 7

Isoflavones of the present invention are chronically administered as a food supplement to a population of humans residing in an area contaminated with radionuclides to treat existing damage caused by radiation exposure and/or prevent further damage from radioisotopes transmitted via food consumption and inhalation.

### EXAMPLE 8

Genistein, genistin, daidzein, and mixtures thereof are chronically administered as a food supplement to population residing inan area contaminated with radionuclides to prevent further damage from the already incorporated radioisotopes in different organs and systems such as, bones, bone marrow, respiratory and gastrointestinal systems.

### EXAMPLE 9

Genistein, genistin, daidzein, and mixtures thereof are administered in the emergency situations, such as nuclear power plant accidents, for the subsequent clean-up operations. Radioprotective effects are demonstrated.

It is intended that the foregoing description be only illustrative of the present invention and that the present invention only be limited by the hereinafter appended claims.

What is claimed is:

1. A method of treating or preventing the effects of radiation in a mammal exposed to radiation, said method comprising administering to said mammal a therapeutically effective amount of an isoflavone.

2. The method of claim 1 wherein said radiation is selected from the group consisting of an acute lethal dose of ionizing radiation, an acute sub-lethal dose of ionizing radiation, a chronic low-dose of ionizing radiation, an acute lethal dose of non-ionizing radiation, an acute sub-lethal dose of non-ionizing radiation, and a chronic low-dose of non-ionizing radiation.

3. The method of claim 2 wherein said radiation is selected from the group consisting of diagnostic X-rays, radiation therapy in cancer treatment, CAT-scans, mammograms, radionuclide scans, interventional radiological procedures under CT or fluoroscopy guidance, tissue-incorporated radionuclides from ingestion of contaminated food or water, and uncontrolled exposure to ionizing radiation from nuclear weapons, radioactive spills, and/or cosmic radiation.

4. The method of claim 1 wherein said isoflavone is selected from the group consisting of genistein, genistin, daidzein, daidzin, glycitein, glycitin, biochannin A, formononetin, O-desmethylangolensin, and equol, their glucosides and derivatives, and mixtures thereof.

5. The method of claim 1 wherein said isoflavone is administered orally, subcutaneously, intramuscularly, intravenously, transdermally, intranasally, or rectally.

6. The method of claim 5 where said isoflavone is administered orally in the form of a capsule, a tablet, an inhaler, a troche, or a food supplement in the form of a food or beverage.

7. The method of claim 1 wherein said isoflavone is administered chronically.

8. The method of claim 1 wherein said isoflavone is administered within 2 weeks prior to exposure to radiation, during radiation exposure, and/or within 2 weeks following radiation exposure.

9. The method of claim 8 wherein said isoflavone is administered within 4 days prior to radiation exposure, during radiation exposure, and/or within 4 days following radiation exposure.

10. A method of treating or preventing damage to living cells, tissues and organs caused by exposure to radiation, said method comprising administering to a therapeutically effective amount of an isoflavone.

11. The method of claim 10 wherein said radiation is selected from the group consisting of an acute lethal dose of ionizing radiation, an acute sub-lethal dose of ionizing radiation, a chronic low-dose of ionizing radiation, an acute lethal dose of non-ionizing radiation, an acute sub-lethal dose of non-ionizing radiation, and a chronic low-dose of non-ionizing radiation.

12. The method of claim 11 wherein said radiation is selected from the group consisting of diagnostic X-rays, radiation therapy in cancer treatment CAT-scans, mammograms, radionuclide scans, interventional radiological procedures under CT or fluoroscopy guidance, tissue-incorporated radionuclides from ingestion of contaminated food or water, and uncontrolled exposure to ionizing radiation from nuclear weapons, radioactive spills, and/or cosmic radiation.

13. The method of claim 10 wherein said isoflavone is selected from the group consisting of genistein, genistin, daidzein, daidzin, glycitein, glycitin, biochannin A, formononetin, O-desmethylangolensin, and equol, their glucosides and derivatives, and mixtures thereof.

14. The method of claim 10 wherein said isoflavone is administered chronically.

15. The method of claim 10 wherein said isoflavone is administered within 2 weeks prior to exposure to radiation, during radiation exposure, and/or within 2 weeks following radiation exposure.

16. The method of claim 15 wherein said isoflavone is administered within 4 days prior to radiation exposure, during radiation exposure, and/or within 4 days following radiation exposure.

17. A method of protecting personnel exposed to radioactive substances, said method comprising administering to said personnel a therapeutically effective amount of an isoflavone.

18. The method of claim 17 wherein said radiation is selected from the group consisting of an acute lethal dose of ionizing radiation, an acute sub-lethal dose of ionizing radiation, a chronic low-dose of ionizing radiation, an acute lethal dose of non-ionizing radiation, an acute sub-lethal dose of non-ionizing radiation, and a chronic low-dose of non-ionizing radiation.

19. The method of claim 18 wherein said radiation is selected from the group consisting of diagnostic X-rays, radiation therapy in cancer treatment, CAT-scans, mammograms, radionuclide scans, interventional radiological procedures under CT or fluoroscopy guidance, tissue-incorporated radionuclides from ingestion of contaminated food or water, and uncontrolled exposure to ionizing radiation from nuclear weapons, radioactive spills, and/or cosmic radiation.

20. The method of claim 17 wherein said isoflavone is selected from the group consisting of genistein, genistin, daidzein, daidzin, glycitein, glycitin, biochannin A, formononetin, O-desmethylangolensin, and equol, their glucosides and derivatives, and mixtures thereof.

21. The method of claim 17 wherein said isoflavone is administered orally, subcutaneously, intramuscularly, intravenously, transdermally, intranasally, or rectally.

22. The method of claim 21 where said isoflavone is administered orally in the form of a capsule, a tablet, an inhaler, a troche, or a food supplement in the form of a food or beverage.

23. The method of claim 17 wherein said isoflavone is administered chronically.

24. The method of claim 17 wherein said isoflavone is administered within 2 weeks prior to exposure to radiation, during radiation exposure, and/or within 2 weeks following radiation exposure.

25. The method of claim 24 wherein said isoflavone is administered within 4 days prior to radiation exposure, during radiation exposure, and/or within 4 days following radiation exposure.

26. A method for increasing survivability of mammals from a lethal dose of radiation, said method comprising administering to said mammal before, during and/or after said lethal dose of radiation a therapeutically effective amount of a compound of the formula: 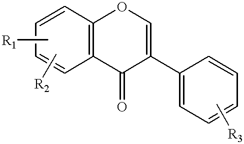
wherein R₁, R₂ and R₃ are independently selected from the group consisting of hydrogen, hydroxyl and alkoxy.

27. The method for increasing survivability of mammals from a lethal dose of radiation as defined in claim 26 wherein said compound is genistein.

28. A method for increasing survivability of mammals from a lethal dose of radiation as defined in claim 12 wherein said compound is administered to said mammal during the time period of approximately 4 days prior to radiation exposure to approximately 4 days subsequent to said lethal dose of irradiation.
